(19) United States
(12) Patent Application Publication
(10) Pub. No.: US 2021/0299376 A1
(43) Pub. Date: Sep. 30, 2021

(54) PROXIMITY-BASED REMOTE VIEWING AND CONTROL OF A VENTILATOR

(71) Applicant: Covidien LP, Mansfield, MA (US)

(72) Inventors: Ryan Gleim, Vista, CA (US); Matthew J. Phillips, Carlsbad, CA (US)

(73) Assignee: Covidien LP, Mansfield, MA (US)

(21) Appl. No.: 17/173,367

(22) Filed: Feb. 11, 2021

Related U.S. Application Data
(60) Provisional application No. 62/994,670, filed on Mar. 25, 2020.

(51) Int. Cl. A61M 16/00; H04W 4/80; H04W 76/10; G06F 3/0484; G06F 3/0488; H04W 4/029; G16H 20/40; G16H 40/67

(52) U.S. Cl. CPC A61M 16/024; A61M 2205/6018; H04W 76/10; G06F 3/04847; G06F 3/0488; H04W 4/029; G16H 20/40; G16H 40/67; A61M 2205/505; A61M 2205/84; A61M 2205/3306; A61M 2205/3584; A61M 2205/3334; A61M 2205/3379; A61M 2205/80; A61M 2205/6063; H04W 4/80

(57) ABSTRACT

Methods and systems for remotely controlling one or more ventilators with a portable device. The portable device may receive a first proximity indication for a first ventilator of a plurality of ventilators, and based on receiving the first proximity indication, establish a first wireless connection between the device and the first ventilator. In addition, the device may receive and display ventilator data regarding ventilation of a patient with the first ventilator. Further, the device may receive a first touch input for a first alteration to a ventilator setting. A signal to change the setting may be transmitted to the ventilator.

### CROSS-REFERENCE TO RELATED APPLICATIONS

This application claims the benefit of U.S. Provisional Application No. 62/994,670, filed Mar. 25, 2020, the complete disclosure of which is hereby incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, and modern ventilators can be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition. To change modes and the settings therein, medical care professionals must interact directly with the ventilator.

### SUMMARY

Aspects of the present disclosure relate to remotely controlling a ventilator with a portable device when the portable device is in proximity to the ventilator. In an aspect, the technology relates to a device for remotely controlling a ventilator. The device includes a display capable of receiving touch input, a processor; and memory storing instructions that, when executed by the processor, causes the device to perform a set of operations. The set of operations include receiving a first proximity indication for a first ventilator of a plurality of ventilators, based on receiving the first proximity indication, establishing a first wireless connection between the device and the first ventilator, and receiving, via the first wireless connection, ventilator data regarding ventilation of a patient with the first ventilator. The set of operations further include displaying the received ventilator data on the display, receiving a first touch input, via the display, for a first alteration to a ventilator setting, and based on the received first touch input, transmitting, via the first wireless connection, a first signal to the first ventilator to alter the ventilator setting.

In an example, the set of operations further include receiving a second proximity indication for a second ventilator of the plurality of ventilators, based on receiving the second proximity indication, establishing a second wireless connection between the device and the second ventilator, receiving a second touch input, via the display, for a second alteration to the ventilator setting, and based on the received second touch input, transmitting, via the second wireless connection, a second signal to the first ventilator to alter the ventilator setting. In another example, the first ventilator is located in a first room of a medical facility, the second ventilator is located in a second room of the medical facility, the first proximity indication is received when the device is in the first room of the medical facility, and the second proximity indication is received when the device is in the second room of the medical facility. In yet another example, the device further comprises a camera, and the first proximity indication is based on an image captured by the camera. In still another example, the captured image is an image of an optical identifier located on at least one of the first ventilator or a structure of a room in which the first ventilator is located. In an additional example, the first proximity indication is a radio-frequency identifier.

In another example, the wireless connection is one of a BLUETOOTH-based connection or a WIFI-based connection. In yet another example, the ventilator setting is one of an inhalation flow setting, a respiratory rate setting, a tidal volume setting, or a positive end-expiratory pressure (PEEP) setting. In still another example, the operations further comprise, replicating, on the display, ventilator data displayed on a screen of the first ventilator.

In another aspect, the technology relates to a method for providing ventilation. The method includes providing, by a first ventilator, ventilation to a first patient according to a first ventilator setting, receiving, by a portable device, a first proximity indication indicating proximity of the portable device to the first ventilator, based on receiving the first proximity indication, establishing a first wireless connection between the device and the first ventilator, receiving, by the portable device via the first wireless connection, ventilator data regarding ventilation of the first patient, displaying, on a display of the portable device, the received ventilator data regarding ventilation of the first patient, receiving, by the portable device, a first input for an alteration to the first ventilator settings, and based on the received first input, transmitting, by the portable device via the first wireless connection, a first signal to the first ventilator to alter the first ventilator setting. The method further includes receiving, by the first ventilator, the first signal, based on receiving the first signal, altering the first ventilation setting, and providing, by the first ventilator, ventilation to the first patient based on the altered first ventilation setting.

In an example, the method further includes providing, by a second ventilator, ventilation to a second patient according to second ventilator setting, receiving, by the portable device, a second proximity indication indicating proximity of the portable device to the second ventilator, based on receiving the second proximity indication, establishing a second wireless connection between the device and the second ventilator, receiving, by the portable device, a second input for an alteration to the second ventilator setting, and based on the received second input, transmitting, by the portable device via the second wireless connection, a second signal to the second ventilator to alter the second ventilator setting. The method further includes receiving, by the second ventilator, the second signal, based on receiving the second signal, altering the second ventilation setting, and providing, by the second ventilator, ventilation to the second patient based on the altered second ventilation settings. In another example, the first input is one of a touch input or a voice input. In yet another example, the method further includes capturing, by the portable device, an image of an optical identifier located on the first ventilator, and the first proximity indication is based on the optical identifier.

In another example, the method further includes receiving, by the portable device, a radio-frequency identification (RFID) signal from one of the first ventilator, an RFID tag located on the first ventilator, or an RFID tag located in a room in which the first ventilator is located, and wherein the first proximity indication is based on the received RFID signal. In yet another example, the method further comprises, based on the first wireless connection being established, locking, by the first ventilator, local changes to ventilator settings. In still another example, the method includes generating, by the first ventilator, sensor data from a plurality of sensors of the first ventilator, determining a first set of sensor data processing operations that require computing resources greater than a computing threshold, determining a second set of sensor data processing operations that require computing resources lower than the computing threshold, performing the first set of sensor data processing operations on the portable device, and performing the second set of sensor data processing operations.

In another aspect, the technology relates to a system for providing ventilation to a plurality of patients with a plurality of ventilators. The system includes a first ventilator of the plurality of ventilators, a second ventilator of the plurality of ventilators, and a portable device. The portable device includes a display, a processor, and memory storing instructions that, when executed by the processor, cause the device to perform a set of operations. The set of operations include receiving a first proximity indication for the first ventilator, based on receiving the first proximity indication, establishing a first wireless connection between the portable device and the first ventilator, receiving a first input, via the display, for an alteration to a first ventilator setting of the first ventilator, and based on the received first input, transmitting, via the first wireless connection, a first signal to the first ventilator to alter the ventilator setting, receiving a second proximity indication for the second ventilator, based on receiving the second proximity indication, establishing a second wireless connection between the portable device and the second ventilator, receiving a second input for an alteration to a second ventilator setting of the second ventilator, and based on the received second input, transmitting, via the second wireless connection, a second signal to the second ventilator to alter the second ventilator setting.

In an example, the set of operations further include, based on receiving the second proximity indication, ceasing the first wireless connection. In another example, the first proximity indication is based on location data for the portable device. In still another example, the first proximity indication is based on a signal strength of a detected beacon signal.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1A depicts a diagram illustrating an example of a ventilator connected to a human patient.

FIG. 1B depicts a diagram illustrating the ventilator of FIG. 1A and a portable device for controlling the ventilator.

FIG. 1C depicts a schematic diagram illustrating features of the portable device.

FIG. 2A depicts an example system for controlling a plurality of ventilators.

FIG. 2B depicts another example system for controlling a plurality of ventilators.

FIGS. 3A and 3B depict an example methods for remotely controlling a ventilator.

FIG. 4 depicts an example method for generating a proximity indication.

FIG. 5 depicts an example method for processing ventilator data.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed above, ventilation provided to patients via a ventilator is controlled based, at least in part, on settings and inputs provided by a medical professional. To provide those inputs and settings, however, the medical professional must directly interact with the ventilator, such as by pressing buttons, providing touch inputs, rotating knobs, etc. Directly interacting with the ventilator may present risks to the medical professional as well as the patient. For example, physically touching different ventilators increases the risk of cross-contamination and the potential spread of diseases. In addition, direct interaction with a ventilator requires physical access to the ventilator, which can be a challenge particularly in the case of patients with highly contagious diseases that may be quarantined.

The present technology looks to alleviate some of those problems by providing a remote control of the ventilator by a portable device. The portable device may be configured to detect the proximity of the portable device to a particular ventilator. Based on the detected proximity, a wireless connection between the portable device and the ventilator may be established. Data may then be exchanged, via the wireless connection, between the portable device and the ventilator. Based on the exchanged data, ventilatory data regarding ventilation of the patient may be displayed on the portable device, and inputs received by the portable device may be transmitted to the ventilator to change the settings of the ventilator. Accordingly, the ventilator may be remotely controlled while the portable device and the medical professional are in proximity of the ventilator. By being proximate to the ventilator, the medical professional is still able to visually monitor the patient, but the medical professional does not have to physical interact with the ventilator itself. In addition, by basing the control of the ventilator on proximity to the device, the medical professional may use a single portable device to control multiple ventilators.

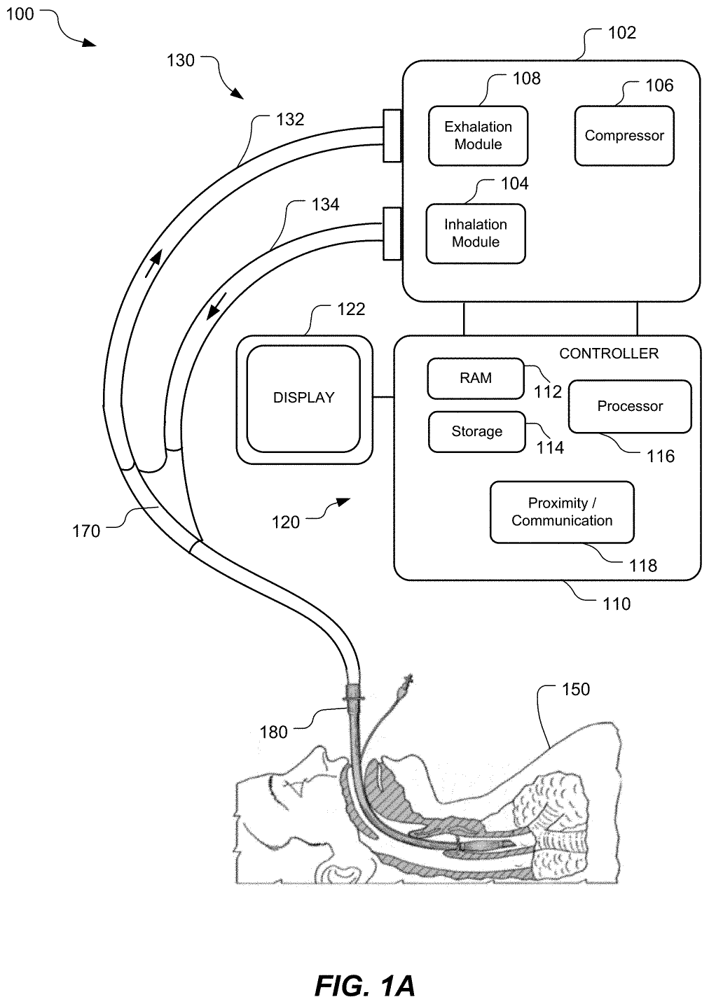

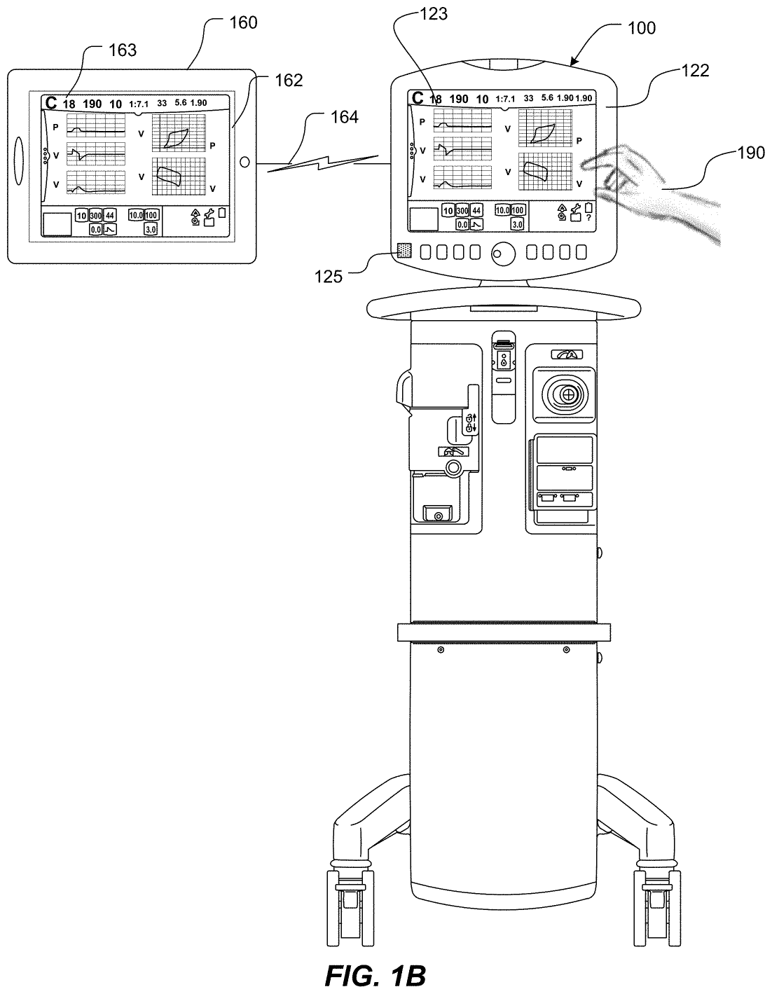

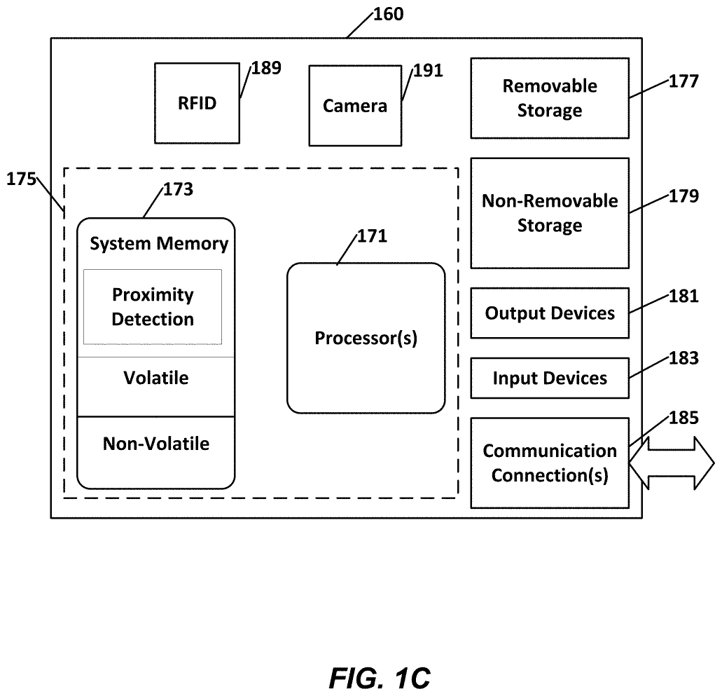

FIG. 1A is a diagram illustrating an example of a ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc.

Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote devices, such as a portable device, may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

FIG. 1B depicts a diagram illustrating the ventilator 100 of FIG. 1A and a portable device 160 for controlling the ventilator 100 and/or viewing data from the ventilator 100. The display 122 of the ventilator is communicatively coupled to the remainder of the ventilator components, such as memory, processors, sensors, etc. The display 122 provides various input screens, for receiving input, and various display screens, for presenting useful information. Inputs may be received from a clinician 190. The display 122 is configured to display a graphical user interface (GUI) 123. The GUI 123 may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, etc.). The elements may include controls, graphics, charts, tool bars, input fields, icons, etc. Alternatively, other suitable means for providing input may be provided on the ventilator 100, for instance by a wheel, keyboard, mouse, or other suitable interactive device. Thus, user interface 123 on the display may accept commands and input through display 122 as touch input or through other input devices. The user interface 123 may also provide useful information in the form of various ventilatory data regarding ventilation of the patient, the physical condition of a patient, and/or a prescribed respiratory treatment. The useful information may be derived by the ventilator 100, based on data collected by sensors, and the useful information may be displayed in the form of graphs, wave representations (e.g., a waveform), pie graphs, numbers, or other suitable forms of graphic display.

The ventilator may control ventilation of a patient 150 according to ventilatory settings. Ventilatory settings may include any appropriate input for configuring the ventilator to deliver breathable gases to a particular patient, including measurements and settings associated with exhalation flow of the breathing circuit. Ventilatory settings may be entered, e.g., by a clinician based on a prescribed treatment protocol for the particular patient, or automatically generated by the ventilator, e.g., based on attributes (i.e., age, diagnosis, ideal body weight, gender, etc.) of the particular patient according to any appropriate standard protocol or otherwise. Ventilatory settings may include inhalation flow, frequency of delivered breaths (e.g., respiratory rate), tidal volume, positive end-expiratory pressure (PEEP), etc.

The portable device 160 also includes a display 162 that is capable of displaying a GUI 163. The GUI 163 may replicate the GUI 123, or a portion thereof, of the ventilator 100. The display 162 may be a touch-screen for receiving inputs and interactions with the GUI 163. In other examples, the portable device 160 may also include other input means, including voice input or through input elements such as buttons, wheels, etc. for inputting data into the portable device 160. The portable device 160 may also establish a wireless connection 164 between the portable device 160 and the ventilator 100. The wireless connection 164 may be any type of wireless connection capable of transmitting data between two devices, such as radio-frequency wireless connections. For example, the wireless connection 164 may be a WIFI-based connection, a BLUETOOTH-based connection, an RF-LITE-based connection, a ZIGBEE-based connection, an ultra-wideband-based connection, and/or an optical connection, such as an infrared-based connection.

The wireless connection 164 may be used to transmit data to the ventilator and/or receive data from the ventilator. For example, data may be transmitted, via the wireless connection 164, from the ventilator 100 to the portable device 160. That transmitted data may be used to populate the GUI 163 of the portable device 160. In addition, data may be transmitted, via the wireless connection 164, from the portable device 160 to the ventilator 100. The transmitted data may be indicative of an input or ventilatory settings change for the ventilator 100. Accordingly, settings for the ventilator may be changed remotely via the portable device 160.

The wireless connection 164 may be initiated or established upon a proximity indication being detected or received. The proximity indication may be an indication that the portable device 160 is in proximity of the ventilator 100. In some examples, the proximity indication may be based on an image captured by the portable device 160. For instance, the portable device 160 may include a camera, and the camera may be used to capture an image of an optical identifier for the ventilator. The optical identifier may be a barcode, such as a two-dimensional barcode or Quick Response (QR) code, for the ventilator 100. When an image of the optical identifier is captured by the portable device 160, the image may be analyzed to extract data from the optical identifier that provides unique information identifying the ventilator 100 from other ventilators. The extracted data may also provide connection information for establishing the wireless connection 164, such as an internet protocol (IP) address, media access control (MAC) address, pairing information, etc. In other examples, the proximity indication may be based on data from a radio-frequency identification (RFID) tag. For instance, the portable device 160 may include an RFID reader and/or writer, such as near-field connection (NFC) capabilities. The RFID reader of the portable device 160 may be used to read an RFID tag that identifies the ventilator 100, similar to how an optical identifier may be used to identify the ventilator 100. For example, the data provided by the RFID tag may include a unique identifier for the ventilator 100 and may also include connection information for establishing the wireless connection.

As an example, the ventilator 100 may include an identifier 125. The identifier 125 may be an optical identifier and/or an RFID tag. Accordingly, a medical professional may approach the identifier 125 with the portable device 160 and capture an image of the identifier 125 and/or read the RFID tag in the identifier 125. While the identifier 125 is depicted as attached to the ventilator 100, in other examples, the identifier 125 may be located in other positions. For instance, the identifier 125 may be attached to another fixture or structure in or near the room of the ventilator. As an example, the identifier 125 may be attached to the placard identifying the room number. Accordingly, an image-capture or reading of the identifier 125 may be accomplished prior to entering the room where the ventilator 100 is located. In other example, the identifier 125 may be an optical identifier that is displayed on the display 122 of the ventilator 100. In further examples, the ventilator itself may emit an RFID signal that is similar to an RFID signal or data that is transmitted from an RFID tag when read or scanned.

In other examples, the proximity indication may be based on the strength of a radio-frequency signal emitted from the ventilator 100. For instance, the ventilator 100 may emit a beacon signal, which may be based on WIFI, BLUETOOTH, or other protocols. The portable device 160 detects that signal and determines a proximity, or relative proximity, to the ventilator 100. As an example, the portable device 160 may be in a medical facility with a plurality of ventilators. Each ventilator 100 may emit a beacon signal, and all or a subset of those beacon signals may be detected by the portable device 160. The portable device 160 may then analyze the strength of each signal to determine which ventilator 100 is closest, or most proximate, to the portable device 160.

In yet other examples, the proximity indication may be based on a location of the portable device 160 as determined by positioning components of the portable device 160, such as positioning or location information from a global positioning system (GPS) of the portable device 160. The location information provided by the GPS system may be compared to the location of plurality of ventilators, and the closest ventilator may be identified.

In other examples, the proximity indication may be based on a manual entry received by the portable device 160. For instance, a medical professional may enter the room number or a unique identifier for the ventilator 100 into the portable device 160. The unique identifier for the ventilator may be provided on the identifier 125. That identification information received by the portable device 160 may be the proximity indication.

The proximity indication may also be based on a selection of a particular ventilator from a user interface presented on the display 162 of the portable device 160. For example, a list or set of selectable user interface elements may be displayed. Each of the selectable user interface elements may correspond to a different ventilator in a plurality of ventilators. For instance, in a hospital environment, each of the ventilators in the hospital may be listed or displayed as different selectable user interface elements. The medical professional using the portable device 160 may select the user interface element corresponding to the most proximate ventilator. That selection may be the proximity indication or a proximity indication may be generated based on the selection.

In some examples, the display of user interface elements corresponding to the different ventilators may be augmented or adjusted by other data or information received by the portable device 160. For instance, based on location data, such as from GPS, the ordering of user interface elements may be updated. As an example, based on the location data, the ventilators that are closest to the portable device 160 may be listed first or otherwise highlighted to make the closest ventilators stand out. As another example, the beacon signals emitted from the ventilators may be used to update the display of user interface elements. For instance, ventilators with the strongest detected beacon signals may be listed first or otherwise highlighted. Additionally or alternatively, ventilators that are not in proximity to the portable device may be removed from the display or otherwise be unelectable. For instance, only ventilators where a beacon signal is detected may be listed for selection. In other examples, only ventilators that have a beacon signal above a strength threshold may be listed for selection. In other examples based on location information from a GPS system, only ventilators within a certain distance threshold may be listed. The medical professional may select which information may be used to augment or adjust the display of user interface elements corresponding to the different ventilators.

FIG. 1C depicts a schematic diagram illustrating features of the portable device 160. In some examples, portable device 160 may be a tablet, smartphone, or other type of portable computing device. In its most basic configuration, the portable device typically includes at least one processor 171 and memory 173. Depending on the exact configuration and type of computing device, memory 173 (storing, among other things, instructions to perform the proximity, control, and display methods disclosed herein) can be volatile (such as RAM), non-volatile (such as ROM, flash memory, etc.), or some combination of the two. This most basic configuration is illustrated in FIG. 1C is indicated by dashed line 175. Further, portable device 160 may also include storage devices (removable, 177, and/or non-removable, 179) including, but not limited to, solid-state devices, magnetic or optical disks, or tape. Similarly, portable device 160 can also have input device(s) 183 such as touch screens, keyboard, mouse, pen, voice input, etc., and/or output device(s) 181 such as a display, speakers, printer, etc. Also included in the environment can be one or more communication connections 185, such as LAN, WAN, point to point, Bluetooth, RF, etc.

In addition, the portable device may include a camera 191. The camera 191 may include the lenses, sensors, and image processing components needed to generate a captured image from a camera. The portable device 160 may also include an RFID reader and/or writer 189 that is capable of reading data from an RFID tag, such as ventilator identification data as discussed above.

Portable device 160 typically includes at least some form of computer readable media. Computer readable media can be any available media that can be accessed by processor 171 or other devices within the portable device 160. By way of example, and not limitation, computer readable media can comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, solid state storage, or any other tangible and non-transitory medium which can be used to store the desired information.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The portable device 160 can be a single computing device operating in a networked environment using logical connections to one or more remote computers. The remote computer can be a personal computer, a server, a router, a network PC, a peer device or other common network node, and typically includes many or all of the elements described above as well as others not so mentioned. The logical connections can include any method supported by available communications media. Such networking environments may be commonplace in hospitals, offices, enterprise-wide computer networks, intranets, and the Internet.

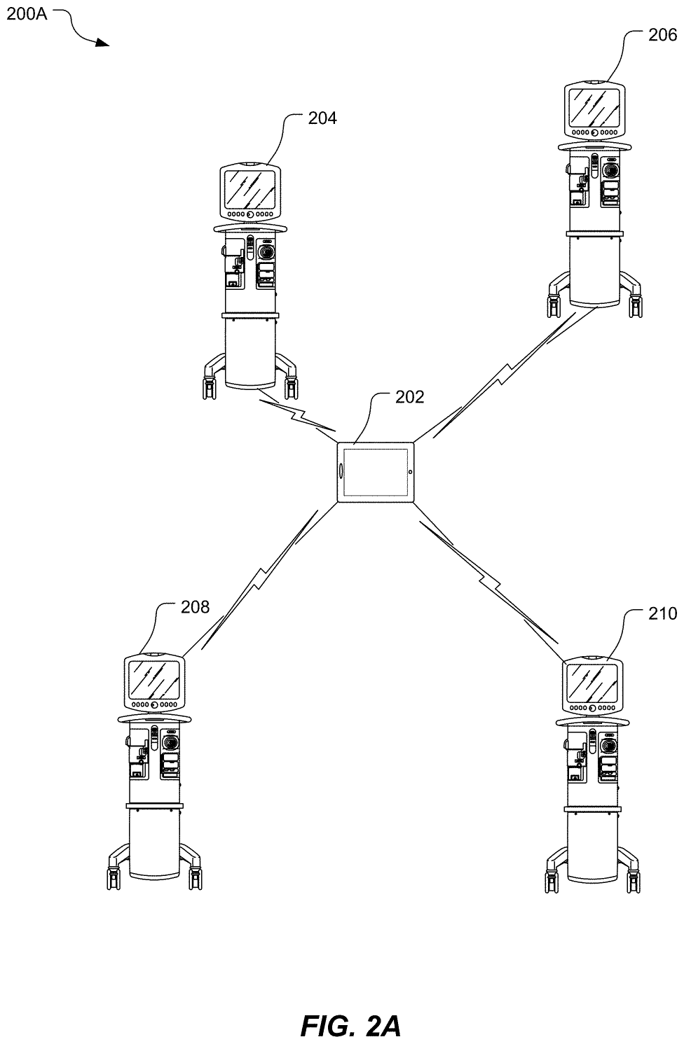

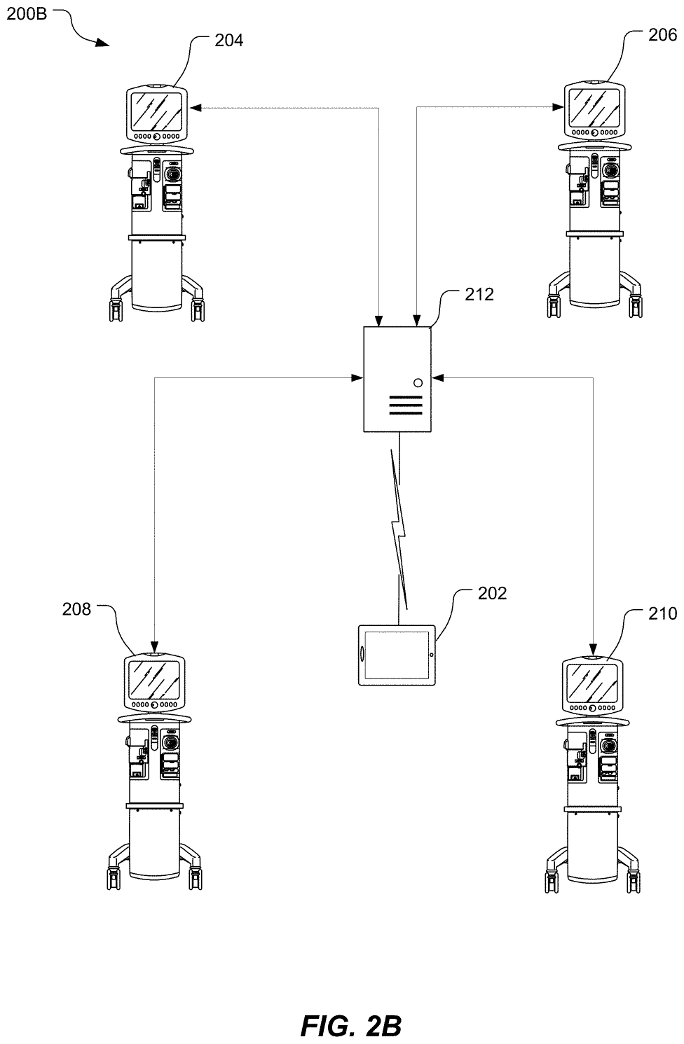

FIG. 2A depicts an example system 200A for controlling a plurality of ventilators 204-210. The system 200A includes a plurality of ventilators 204-210, including a first ventilator 204, a second ventilator 206, a third ventilator 208, and a fourth ventilator 210. The system 200A also includes a portable device 202. The portable device 202 is capable of establishing a wireless connection directly with each of the ventilators 204-210. In some examples, the portable device 202 may maintain a wireless connection between multiple ventilators 204-210. In other examples, the portable device 202 may maintain only one wireless connection at a time.

As discussed above, the wireless connection(s) may be established or initiated based on the detection of a proximity indication. For example, the portable device 202 may receive a proximity indication that indicates the first ventilator 204 is the most proximate ventilator to the portable device 202. Upon receiving that proximity indication, the portable device 202 may establish a direct wireless connection between the portable device 202 and the first ventilator 204. At a later time when the portable device 202 has moved closer to the second ventilator 206, the portable device 202 may receive a new proximity indication that indicates the second ventilator 206 is now the most proximate ventilator to the portable device 202. In response to that new proximity indication, the portable device 202 may establish a new wireless connection between the portable device 202 and the second ventilator 206. In addition, the portable device 202 may cease or end the wireless connection between the portable device 202 and the first ventilator 204. In a similar manner, new or additional wireless connections may be made between the portable device 202 and the third ventilator 208 and fourth ventilator 210 as the portable device 202 moves into proximity of those ventilators.

FIG. 2B depicts another example system 200B for controlling the plurality of ventilators 204-210. System 200B is substantially similar to system 200A with the exception of how the plurality of ventilators 204-210 may communication with the portable device. More specifically, in addition to the plurality of ventilators 204-210 and the portable device 202, the system 200B may also include a server 212. Each of the ventilators 204-210 may have a connection to the server 212. The connections to the server 212 may be wired or wireless. For instance, the plurality of ventilators 204-210 may be connected to the server 212 via an Ethernet connection or be connected via wireless internet as part of a local-area network (LAN) or wide-area network (WAN). As should be appreciated, switches and/or routers (not depicted), along with other potential networking hardware, may be present in system 200B to facilitate the connections of the ventilators 204-210 to the server 212.

The portable device 202 may also be wirelessly connected to the server 212. The wireless connection to the server 212 may be facilitated through WIFI as part of a LAN or WAN connected to the server 212. In the system 200B, the connection between the portable device 202 and each of the ventilators 204-210 is facilitated through the server 212 by utilizing the server's connections to each of the ventilators 204-210. For example, when the portable device 202 is in proximity of the first ventilator 204 and receives or detects a proximity indication, a connection between the portable device 202 and the first ventilator 204 is established via the server 212. The connection may be wireless if the first ventilator 204 is wirelessly connected to the server 212. If the first ventilator 204 is connected to the server 212 via a wired connection, the connection established between the portable device 202 and the first ventilator 204 may be partially wireless and partially wired. The connection formed may be a session between the portable device 202 and the first ventilator 212 as set forth in the transmission control protocol (TCP), or other similar methods or processes for establishing a connection between two computing devices in a network. As the portable device 202 is moved to different locations, similar connections may be established between the portable device 202 and the second ventilator 206, the third ventilator 208, and/or the fourth ventilator 210. While only four ventilators 204-210 and a single portable device 202 are depicted in systems 200A and 200B, it should be appreciated that a greater or fewer number of such devices may be utilized in systems for controlling ventilation.

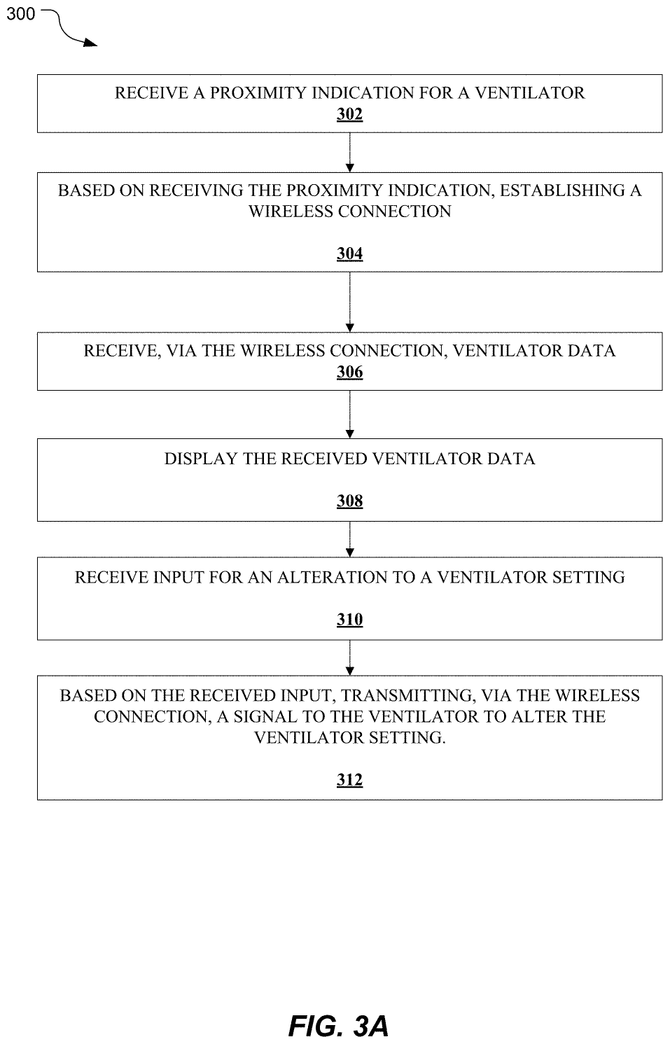

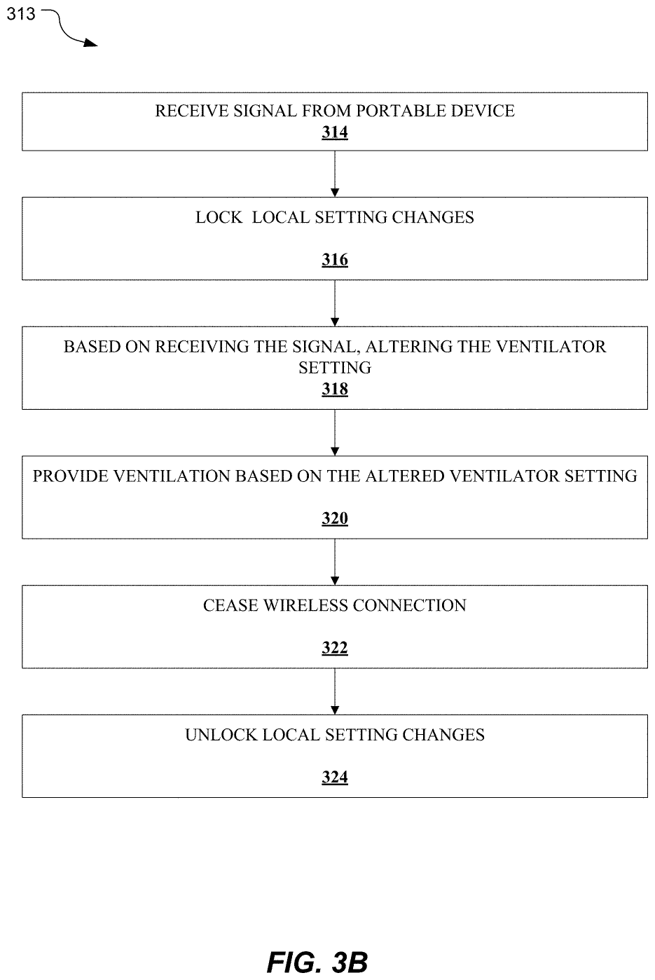

FIG. 3A depicts an example method 300 for remotely controlling a ventilator. The operations of method 300 may be performed by the portable device and/or other components described in the above systems. At operation 302 a proximity indication for a particular ventilator is received or detected. The proximity indication indicates a particular ventilator that is proximate to the portable device. The proximity indication may be any of the proximity indications discussed herein. For example, the proximity indication may be based on a captured image of an optical identifier, a read of an RFID tag, location data for the portable device, and/or detection of a beacon signal from a ventilator. The proximity indication may also be based on a received selection of a ventilator on a user interface displayed on the portable device.

At operation 304, based on the received or detected proximity indication, a wireless connection is established between the portable device and the ventilator corresponding to the proximity indication. The wireless connection may be a direct connection between the portable device, such as a BLUETOOTH or ZIGBEE connection. In other examples, the wireless connection may be an indirect connection, such as a connection through a server.

At operation 306, ventilatory data is received via the wireless connection established in operation 304. The ventilatory data may be received from the ventilator to which the portable device is wirelessly connected. The ventilatory data may include data regarding ventilation of the patient with the ventilator. For example, the ventilatory data may include pressure, volume, and flow information that is used by the ventilator to generate to plots, charts, and other information displayed on the user interface of the ventilator. The ventilatory data may also include data to replicate the user interface of the ventilator, or a portion thereof, on a display of portable device.

At operation 308, the ventilatory data is displayed on the portable device. Displaying the ventilatory data on the portable device may include replicating at least a portion of the user interface displayed on the display of the ventilator. For instance, plots, charts, and/or values that are displayed on the ventilator may also be displayed on the portable device. In addition or alternatively, the ventilatory data may be displayed in a different format than how the ventilatory data is displayed on the ventilator. For example, a first plot of ventilatory data may be displayed on the portable device and a second, different plot may be displayed on the ventilator. Accordingly, the display of the portable device may be used to compliment or augment the display of data on the ventilator. Ventilator settings and options may also be displayed on the display of the portable device.

At operation 310, an input is received for an alteration to a ventilator setting. The input may be received from a medical professional via a touch of a touch-screen display of the portable device. For example, interactive settings and/or options for ventilation may be displayed in a user interface of the portable device. A medical professional may interact with such user interface features to change or alter a ventilator setting. At operation 312, based on the input received in operation 310, a signal is generated and transmitted from the portable device to the ventilator via the wireless connection established in operation 304. The signal may be a digital signal that indicates the setting or option to be changed and a value or values for the setting or option.

FIG. 3B depicts another method 313 for remotely controlling a ventilator. The operations of method 313 may be performed by a ventilator and/or other components described in the systems above. At operation 314, a signal for changing ventilator settings is received by a ventilator. The received signal may be the signal generated by the portable device at operation 312 of method 300 depicted in FIG. 3A. In such examples, the signal may be received via the wireless connection established between the portable device and the ventilator in operation 304 of method 300 depicted in FIG. 3A.

At operation 316, local setting changes may be locked. For example, in response to receiving a signal indicating that a change to ventilator settings should be made, local changes to settings via physical interaction with the ventilator may be prevented or locked. In other examples, local changes to settings may be locked upon or based on the wireless connection being established in operation 304 of method 300 depicted in FIG. 3A. In other examples, the setting changes may be locked in response to a user selection of a user interface to lock the local settings. For instance, a selectable user interface element may be presented on the user interface of the portable device and/or the ventilator. Based on receiving a selection of that user interface element, the ventilator may lock or prevent changes to the ventilator settings. By locking or preventing local changes, conflicting changes to ventilator settings are prevented, such as in the situation where one person is interacting with the portable device and another person is physically interacting with the ventilator. The local settings changes may remain locked for as long as the wireless connection between the portable device and the ventilator is established.

At operation 318, based on the signal received in operation 314, the ventilator setting is altered. For example, as discussed above, the signal may indicate the type of setting or option that is to be changed as well as the value to which the setting should be altered. Thus, upon receiving the signal, the ventilator may parse or analyze the signal to determine the setting to change and the value for the corresponding setting. The ventilator may then make that change or alteration as part of operation 318. In operation 320, ventilation is provided to the patient based on the adjusted ventilation settings.

At operation 322, the wireless connection between the portable device and the ventilator is ceased, ended, or otherwise disconnected. The operation of ceasing the wireless connection may be performed and/or initiated by the ventilator and/or the portable device. For example, a selectable user interface option to end the wireless connection or disconnect the portable device from the ventilator may be displayed on the user interface of the portable device and/or the ventilator. When a selection of the user interface option is received on the either the ventilator or the portable device, the wireless connection is ceased. The wireless connection may also cease when the portable device is no longer in proximity with the ventilator. For example, if the portable device moves a threshold distance away from the ventilator, operation 322 may be triggered. The distance from the ventilator may be based on GPS-based location data or an analysis of signal strength of the beacon signal emitted from the ventilator. In other examples, the wireless connection may end after a time-out period. For example, after the wireless connection is established, the wireless connection may be automatically ceased one a certain time period expires. The time period may expire or elapse based on a frequency of interaction with the portable device by the medical professional for the duration of the time period. For example, the time period may be set to only cease if there is no interaction with the portable device by the medical profession during the time period. In another example, if an interaction does occur, the time period may be restarted or renewed.

At operation 324, the changes to the local settings are unlocked or otherwise allowed to occur via physical interaction with the ventilator. Unlocking the ability to locally change the settings may be performed based on or upon the wireless connection ceasing at operation 322. In other examples, unlocking the ability to locally change ventilator may be performed upon or based on receiving a selection of a user interface element presented on the portable device and/or the ventilator. For example, a selectable user interface element may be displayed on the user interface of the portable device and/or the ventilator. A user may select that user interface element to unlock the ability to make local changes to the ventilator settings. In an example where that user interface element is selected on the portable device, operation 324 may be performed prior to the wireless connection being ceased in operation 322.

While the methods 300 and 313 in FIGS. 3A-3B are discussed with reference to a single ventilator, it should be appreciated that methods may be applied for multiple ventilators. As an example, the methods described above and herein may be first performed for a first ventilator of a plurality of ventilators, such as a plurality of ventilators located in a hospital or other medical facility. The methods may then be performed for a second ventilator in the plurality of ventilators. For instance, when the portable device is in proximity of the second ventilator, a wireless connection may be established between the portable device and the second ventilator. The second ventilator may then be remotely controlled via the portable device.

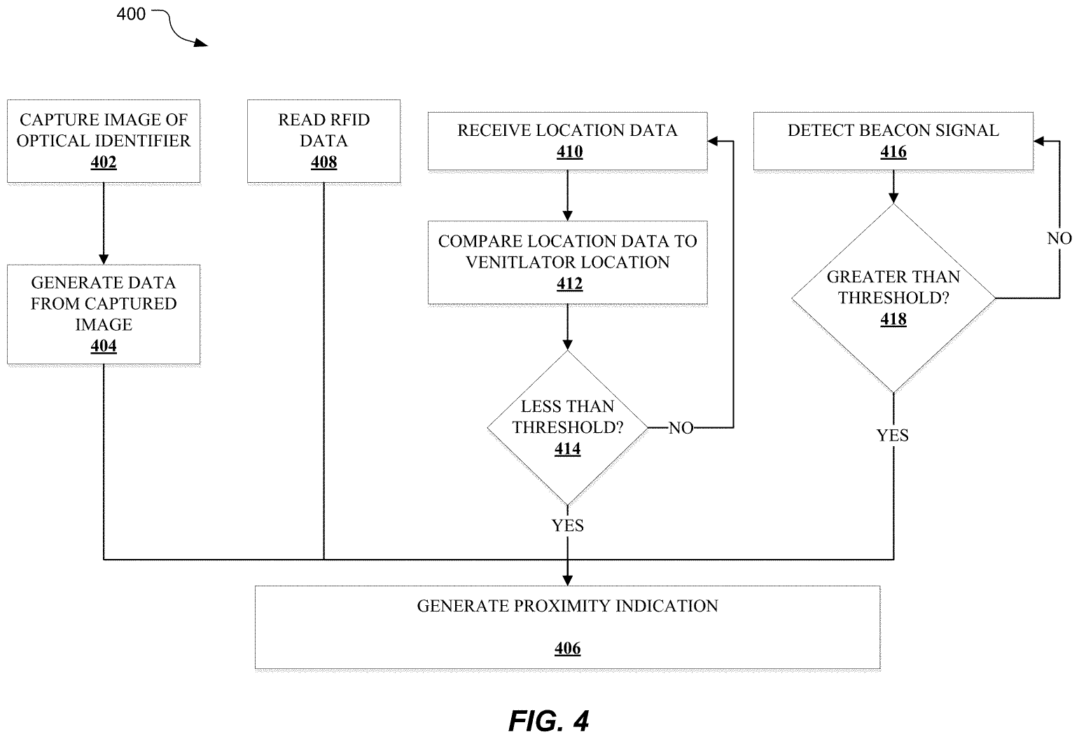

FIG. 4 depicts a method 400 for generating a proximity indication. The proximity indication may be the proximity indication that is received or detected in operation 304 of method 300 depicted in FIG. 3A. The operations of method 400 may be performed by the portable device and/or the ventilator. The method 400 depicts multiple manners for generating a proximity indication. However, in practice not all manners may be used to generate the proximity indication.

At operation 402, an image of an optical identifier may be captured. The image may be captured by a camera of the portable device. The optical identifier may be a barcode, such as a two-dimensional barcode or Quick Response (QR) code, for the ventilator. The optical identifier may be affixed to or otherwise integrated into the ventilator. The optical identifier may also be displayed on a display screen of the ventilator. In other examples, the optical identifier may be affixed to a structure in or just outside the room in which the ventilator is located.

At operation 404, data from the captured image is generated. The data may be based on the barcode or QR code of the optical identifier, and the generated data may include unique information identifying a particular ventilator from other ventilators. The generated data may also provide connection information for establishing the wireless connection between the portable device and the ventilator. For instance the connection information may include data such as an internet protocol (IP) address, a media access control (MAC) address, pairing information, etc. The generated data may also include an indication that an optical identifier has, in fact, been captured in in the image.

At operation 406, a proximity indication is generated. The proximity indication may be based on the captured image in operation 402 and/or the generated data in operation 404. For example, the detection of the optical identifier in the captured image may cause the generation of the proximity indication. The proximity indication may be a software indication, notification, or call and/or also may include software operations such as setting a flag or variable to indicate that the portable device is in proximity of the ventilator. The proximity indication may also include data generated from the captured image, such as the connection information.

The proximity indication generated in operation 406 may also, or alternatively, based on a read of RFID tag. For example, at operation 408, an RFID tag may be read by the portable device. The RFID tag may be affixed to or otherwise integrated into the ventilator. In other examples, the RFID tag may be affixed to a structure in or just outside the room in which the ventilator is located. When the RFID tag is read, the information or data that is read from the RFID tag may include some of the same information as the data generated in operation 404 from the captured image of the optical identifier. For example, the data read from the RFID tag may include connection information and a/or a unique identifier for the ventilator to which the RFID tag corresponds. The proximity indication generated in operation 406 may then be based on the data read from the RFID tag and/or the occurrence of the RFID tag being read.

The proximity indication may also, or alternatively, be based on location data. For example, location data for the portable device may be received or accessed in operation 410. The location data may be generated by a GPS component of the portable device. At operation 412, the location data for the portable device received in operation 410 is compared to location data for a ventilator and/or a plurality of ventilators. The location data for each ventilator may be static or dynamic. For instance, the location data for each ventilator may be manually entered and stored in a table. Thus, the location data for each ventilator may be retrieved from the table. In other examples, the location data for the ventilator may be generated from location systems, such as GPS components, within each of the ventilators. Comparing the location data of the portable device to the location data of the ventilator or plurality of ventilators allows for a distance between the portable device and each ventilator to be determined.

At operation 414, the distance between the portable device and a particular ventilator is compared to a proximity threshold distance. If the distance between the portable device and the particular ventilator is less than the proximity threshold distance, then the method 400 flows to operation 406 where the proximity indication is generated. The distance between the portable device and the particular ventilator is greater than the proximity threshold distance, the method 400 flows back to operation 410 where location data for the portable device is received and comparisons to ventilator location data is repeated.

The proximity indication may also, or alternatively, be based on detected beacon signal strength. For example, at operation 416 a beacon signal strength from one or more ventilators may be detected. As discussed above, in some examples, a ventilator may emit a beacon signal. The portable device may detect that beacon signal in operation 416. As part of detecting the beacon signal, the portable device may determine a strength of the beacon signal. In some examples, the portable device may also extract additional information from the beacon signal. For instance, the beacon signal may also include data within the signal. For instance, the signal may be Bluetooth-based signal, such as a BLUETOOTH low energy (BLE) signal that includes data, such as a unique identifier for the ventilator generating the signal and/or connection information for connecting to the ventilator. As another example, the beacon signal may include a WIFI beacon frame as defined in the IEEE 80.11 standard. The beacon signal may also be probe request generated from the ventilator. That beacon frame or probe request may include an identifier for the ventilator and/or other connection information for connecting to the ventilator. In some examples, the functionalities between the portable device and the ventilator may be switched. For example, the portable device may emit the beacon signal, and the ventilator may detect the beacon signal.

At operation 418, the signal strength of the beacon signal is compared to a proximity signal strength threshold. If the signal strength of the beacon signal is less than the proximity strength threshold, then the method 400 flows back to operation 416 where the beacon signal continues to be detected. If the signal strength of the beacon signal is greater than the proximity strength threshold, then the method 400 flows to operation 406 where the proximity indication is generated. As should be appreciated from the foregoing, the generation of the proximity indication may be based on any combination of a captured optical identifier, a read of an RFID tag, location data, and/or signal strength analysis.

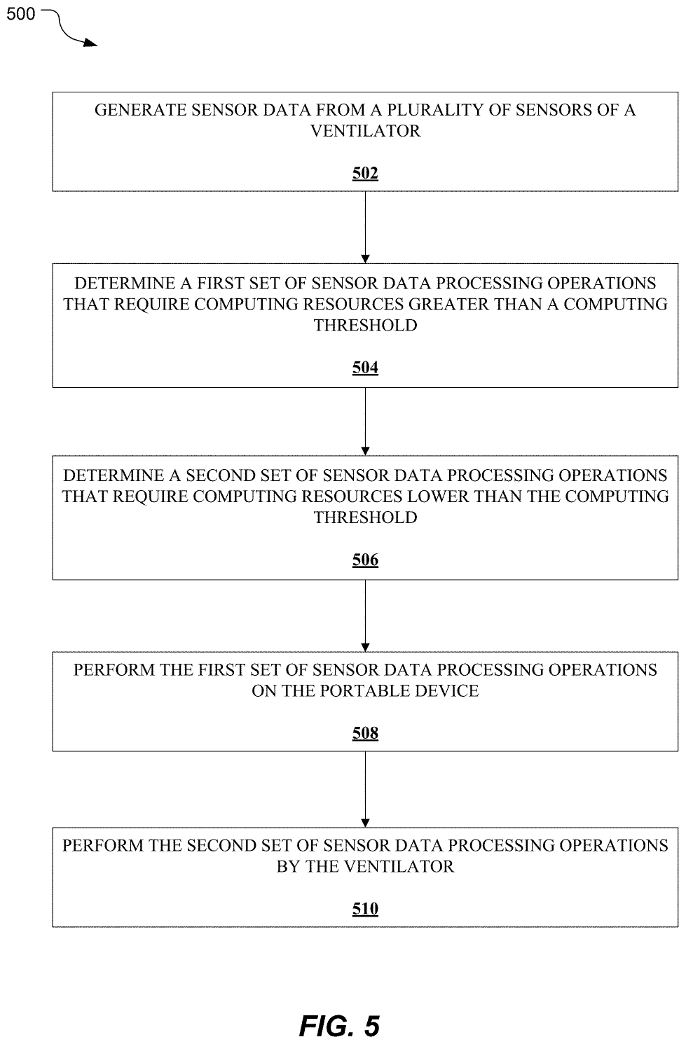

FIG. 5 depicts an example method 500 for processing ventilator data. In some instance, the data generated by ventilator, such as by the various sensors of the ventilator, can be processed to provide additional insights into the ventilation of the patient. A substantial amount of data processing is already completed on the ventilator, such as the data processing required to generate the graphs, plots, and other data that is displayed on the graphical user interface of the ventilator. Some data processing tasks, however, require substantial additional computing resources to be completed. While some of these tasks may be completed on the ventilator, performing those tasks may take away or restrict computing resources that are primarily used for the core ventilation functions of the ventilator. To help prevent such a restriction of resources, but still allow for the processing tasks to be performed, some of the tasks may be offloaded to the portable device when the wireless connection is established between the ventilator and the portable device. Method 500 provides an example method for implementing such functionality.

For example, at operation 502 sensor data is generated from the plurality of sensors on a ventilator. At operation 504, a first set of sensor data processing operations that require computing resources greater than a computing threshold are determined or identified. The computing threshold may be based on required memory, processing time, processing power or speed, among other factors. If a particular task requires an amount of computing resources that is greater than the computing threshold, that particular task is categorized and placed in a first set of tasks.

At operation 506, a second set of sensor data processing operations that require computing resources less than the computing threshold are determined or identified. For example, if a particular task requires an amount of computing resources that is less than the computing threshold, that particular task is categorized and placed in a second set of tasks.

At operation 508, the first set of sensor data processing operations are performed on the portable device. At operation 510, the second set of sensor data processing operations are performed by the ventilator. Accordingly, the processing tasks that require additional computing resources may be performed by a device other than the ventilator, which allows the ventilator to dedicate its resources to the core ventilation features.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Thus, myriad software/hardware/firmware combinations are possible in achieving the functions, features, interfaces, and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A device for remotely controlling a ventilator, the device comprising:
a display capable of receiving touch input;
a processor; and
memory storing instructions that, when executed by the processor, causes the device to perform a set of operations, comprising:
receiving a first proximity indication for a first ventilator of a plurality of ventilators;
based on receiving the first proximity indication, establishing a first wireless connection between the device and the first ventilator;
receiving, via the first wireless connection, ventilator data regarding ventilation of a patient with the first ventilator;
displaying the received ventilator data on the display;
receiving a first touch input, via the display, for a first alteration to a ventilator setting; and
based on the received first touch input, transmitting, via the first wireless connection, a first signal to the first ventilator to alter the ventilator setting.

2. The device of claim 1, wherein the set of operations further comprise:
receiving a second proximity indication for a second ventilator of the plurality of ventilators;
based on receiving the second proximity indication, establishing a second wireless connection between the device and the second ventilator;
receiving a second touch input, via the display, for a second alteration to the ventilator setting; and
based on the received second touch input, transmitting, via the second wireless connection, a second signal to the first ventilator to alter the ventilator setting.

3. The device of claim 2, wherein:
the first ventilator is located in a first room of a medical facility;
the second ventilator is located in a second room of the medical facility;
the first proximity indication is received when the device is in the first room of the medical facility; and
the second proximity indication is received when the device is in the second room of the medical facility.

4. The device of claim 1, wherein:
the device further comprises a camera; and
the first proximity indication is based on an image captured by the camera.

5. The device of claim 4, wherein the captured image is an image of an optical identifier located on at least one of the first ventilator or a structure of a room in which the first ventilator is located.

6. The device of claim 1, wherein the first proximity indication is a radio-frequency identifier.

7. The device of claim 1, wherein the wireless connection is one of a BLUETOOTH-based connection or a WIFI-based connection.

8. The device of claim 1, wherein the ventilator setting is one of an inhalation flow setting, a respiratory rate setting, a tidal volume setting, or a positive end-expiratory pressure (PEEP) setting.

9. The device of claim 1, wherein the operations further comprise, replicating, on the display, ventilator data displayed on a screen of the first ventilator.

10. A method for providing ventilation, the method comprising:
providing, by a first ventilator, ventilation to a first patient according to a first ventilator setting;
receiving, by a portable device, a first proximity indication indicating proximity of the portable device to the first ventilator;
based on receiving the first proximity indication, establishing a first wireless connection between the device and the first ventilator;
receiving, by the portable device via the first wireless connection, ventilator data regarding ventilation of the first patient;
displaying, on a display of the portable device, the received ventilator data regarding ventilation of the first patient;
receiving, by the portable device, a first input for an alteration to the first ventilator settings;
based on the received first input, transmitting, by the portable device via the first wireless connection, a first signal to the first ventilator to alter the first ventilator setting;
receiving, by the first ventilator, the first signal;
based on receiving the first signal, altering the first ventilation setting; and
providing, by the first ventilator, ventilation to the first patient based on the altered first ventilation setting.

11. The method of claim 10, further comprising:
providing, by a second ventilator, ventilation to a second patient according to second ventilator setting;
receiving, by the portable device, a second proximity indication indicating proximity of the portable device to the second ventilator;
based on receiving the second proximity indication, establishing a second wireless connection between the device and the second ventilator;
receiving, by the portable device, a second input for an alteration to the second ventilator setting; and
based on the received second input, transmitting, by the portable device via the second wireless connection, a second signal to the second ventilator to alter the second ventilator setting;
receiving, by the second ventilator, the second signal;
based on receiving the second signal, altering the second ventilation setting; and
providing, by the second ventilator, ventilation to the second patient based on the altered second ventilation settings.

12. The method of claim 10, wherein the first input is one of a touch input or a voice input.

13. The method of claim 10, further comprising:
capturing, by the portable device, an image of an optical identifier located on the first ventilator; and
wherein the first proximity indication is based on the optical identifier.

14. The method of claim 10, further comprising:
receiving, by the portable device, a radio-frequency identification (RFID) signal from one of the first ventilator, an RFID tag located on the first ventilator, or an RFID tag located in a room in which the first ventilator is located; and
wherein the first proximity indication is based on the received RFID signal.

15. The method of claim 10, further comprising, based on the first wireless connection being established, locking, by the first ventilator, local changes to ventilator settings.

16. The method of claim 10, further comprising:
generating, by the first ventilator, sensor data from a plurality of sensors of the first ventilator;
determining a first set of sensor data processing operations that require computing resources greater than a computing threshold;
determining a second set of sensor data processing operations that require computing resources lower than the computing threshold;
performing the first set of sensor data processing operations on the portable device; and
performing the second set of sensor data processing operations.

17. A system for providing ventilation to a plurality of patients with a plurality of ventilators, the system comprising:
a first ventilator of the plurality of ventilators;
a second ventilator of the plurality of ventilators;
a portable device comprising:
a display;
a processor;
memory storing instructions that, when executed by the processor, cause the device to perform a set of operations comprising:
receiving a first proximity indication for the first ventilator;
based on receiving the first proximity indication, establishing a first wireless connection between the portable device and the first ventilator;
receiving a first input, via the display, for an alteration to a first ventilator setting of the first ventilator;
based on the received first input, transmitting, via the first wireless connection, a first signal to the first ventilator to alter the ventilator setting;
receiving a second proximity indication for the second ventilator;
based on receiving the second proximity indication, establishing a second wireless connection between the portable device and the second ventilator;
receiving a second input for an alteration to a second ventilator setting of the second ventilator; and
based on the received second input, transmitting, via the second wireless connection, a second signal to the second ventilator to alter the second ventilator setting.

18. The system of claim 17, wherein the set of operations further comprise, based on receiving the second proximity indication, ceasing the first wireless connection.

19. The system of claim 17, wherein the first proximity indication is based on location data for the portable device.

20. The system of claim 17, wherein the first proximity indication is based on a signal strength of a detected beacon signal.
